(19) United States
(12) Patent Application Publication
(10) Pub. No.: US 2008/0171964 A1
(43) Pub. Date: Jul. 17, 2008

(54) Microwave Ablation Water Jet Scalpel

(76) Inventors: Lin Sheng, Beijing (CN); Chengzhong Chang, Beijing (CN)

Correspondence Address: Liu & Shields LLP, 136-65 37th Ave., Suite 212, Flushing, NY, 11354, US

(21) Appl. No.: 11/869,251

(22) Filed: Oct. 9, 2007

(30) Foreign Application Priority Data
Jan. 16, 2007 (CN) 200710000839.5

(51) Int. Cl. A61B 18/18

(52) U.S. Cl. 604/22; 606/33

(57) ABSTRACT

A microwave ablation water jet scalpel, used for minimally invasive ablation of human tumor, in the present invention combines a microwave scalpel with a liquid-ejection system in which the liquid-ejecting rate is controllable. The improved microwave scalpel allows normal saline to be ejected into the tissue where the scalpel is working. As a result, this relieves many problems associated with high heat generated during the operation of a microwave.

### CROSS-REFERENCE TO RELATED APPLICATIONS

This application claims foreign priority benefits under 35 USC § 119(a) to China Patent Application No. 200710000839.5, filed on Jan. 16, 2007, which is incorporated herein by reference.

### BACKGROUND OF THE INVENTION

1. Field of the Invention

Our invention relates to the medical instrument, to be more specific, it is a microwave ablation water jet scalpel that can be used for minimally invasive ablation of tumors.

2. Background Technology

Malignancy is one of the main threats to human health and life. Currently no medicine is able to cure malignant tumors completely and surgical operations are still the basic approach to remove tumors. Recently, a technology, called minimally invasive treatment of human tumor, has been receiving more and more attentions, because of its high efficiency in inactivating tumor cells in situ and its ability of minimizing trauma and pain to patients and incurring very little complication. The principle behind this technology is to first locate tumor lesion by ultrasound, CT or MRI, and then inject high energy to the lesion area to kill tumor tissues. Majority of minimally invasive treatment equipments utilize radiofrequency, microwave, Argon-Helium cryosurgical ablation, high intensity focused ultrasound, and etc. Compared to other methods, microwave has many advantages, including easy puncture, small trauma, no bone interference and stable ablation shape.

However, high temperature (above 100° C.) caused by operating a microwave scalpel can carbonize surrounding tissues, which prevents microwave from penetrating further into deep tissues and limits the range of ablation area. In addition, the standing wave inside the microwave scalpel and the microwave reflected from the carbonized area around the scalpel blade raise the temperature of the microwave antennas and scald surrounding tissues and skins.

Therefore, it is an object of the present invention to provide a means that overcomes the problems caused by high temperature during operation of a microwave scalpel. These problems include adhesion between the blade of a microwave scalpel and tissues, the carbonization of tissues and small working ablation area.

It is another object of the present invention to provide a means that delivers chemotherapy drugs during operation of a microwave scalpel.

It is a further object of the present invention to provide a means that allows operator of a microwave scalpel to control the temperature of the scalpel.

It is yet another object of the present invention to provide a microwave scalpel that is easily assembled and disassembled.

### SUMMARY OF THE INVENTION

Accordingly, the present invention presents a microwave scalpel attached with a liquid-injection means. The injected liquid could be, but not limited to, saline solution with or without chemotherapy drugs. The liquid-ejection rate is preferably controllable.

Therefore, a microwave ablation water jet scalpel for medical operation comprises:
1. a microwave scalpel component that comprises
   a microwave generating means,
      a handle for hand-holding, and
      a blade that is attached to one end of said handle; and
2. a liquid-manipulation component that comprises
   a liquid-entry means, and
   a liquid-ejection means,
whereby liquid is ejected by said liquid-ejection means to the operating area where said blade is in order to ease the effect caused by heat generated during the operation of said microwave scalpel.

In a preferred embodiment, a microwave scalpel comprises:
1. a microwave generating module that comprises a microwave electrode interface, a conducting wire extending from said interface, and a first assembly frame;
2. an internal module that comprises an internal pipe which contains at least one liquid-outlet, a second assembly frame through which said internal pipe locks into said first assembly frame of said microwave generating module, and a third assembly frame, wherein said conducting wire passes through the inside of said internal pipe;
3. an external module that comprises an external pipe which contains at least one liquid-ejection pore and one liquid-entry pore, a fourth assembly frame through which said external pipe locks into said third assembly frame of said internal pipe, and a fifth assembly frame, wherein a cavity formed between the outside surface of said internal pipe and the inside surface of said external pipe;
4. a scalpel module that comprises a holder, a sixth assembly frame through which said holder locks into said fifth assembly frame of said external pipe, a blade securely attached to said holder, and an insulating tube receiving said conducting wire out from said internal pipe and separating said conducting wire from said blade; and
5. a stopper attached to the outside surface of said internal pipe,
wherein said external pipe is rotatable against said internal pipe, whereby said liquid-ejection pore is blocked by said stopper when said external pipe rotates into a position to stop liquid ejecting from inside of the cavity, and whereby said liquid-ejection pore is unblocked when said external pipe rotates off the position to allow liquid ejecting from inside of the cavity.

On the preferred embodiment, the surface of said handle further contains calibrations and medical insulation coating.

### BRIEF LIST OF REFERENCE NUMERALS UTILIZED IN THE DRAWING

01—electrical outlet core
02—core seat
03—core seal
04—core cushion
05—electrical outlet shell
06—assembly frame of the electrical outlet
07—seal cushion II
08—assembly frame of the internal pipe
09—microwave conducting wire
10—internal pipe
11—seal cushion I
12—assembly frame of the external pipe
13—external pipe
14—weldment (assembly frame and blade holder)
15—isolation tube
16—blade
17—insulation coating
18—water ejection pores
19—stopper on the internal pipe
20—water storage cavity
21—water outlet pore
22—water entry pore
23—wire metal outer tube
24—wire insulation tube

### BRIEF DESCRIPTION OF THE DRAWINGS

A more thorough understanding of the invention may be obtained by a study of the following detailed description taken in connection with the accompanying drawings in which:

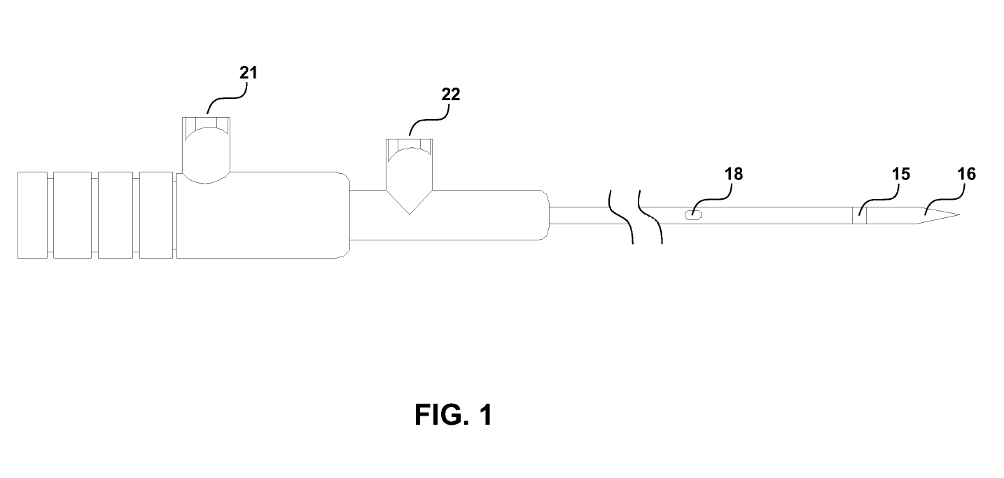

FIG. 1 is the plan view of the microwave ablation water jet scalpel;

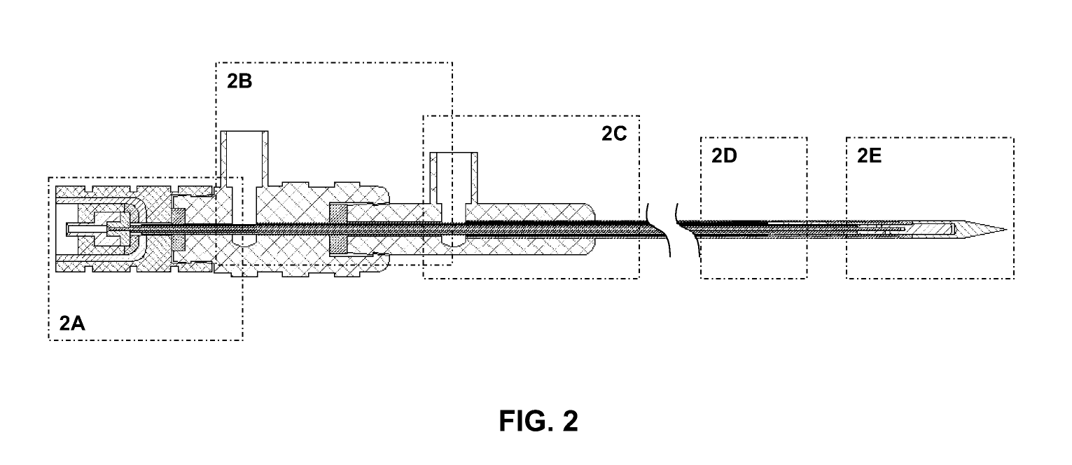

FIG. 2 is a sectional view of the microwave ablation water jet scalpel;

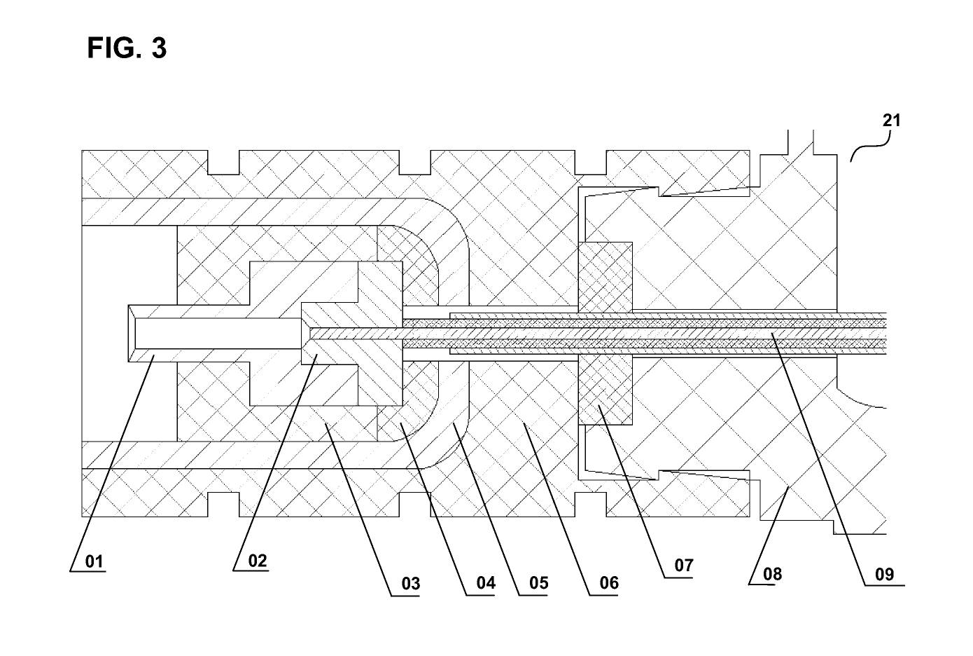

FIG. 3 is a detail view of section 2A in FIG. 2.

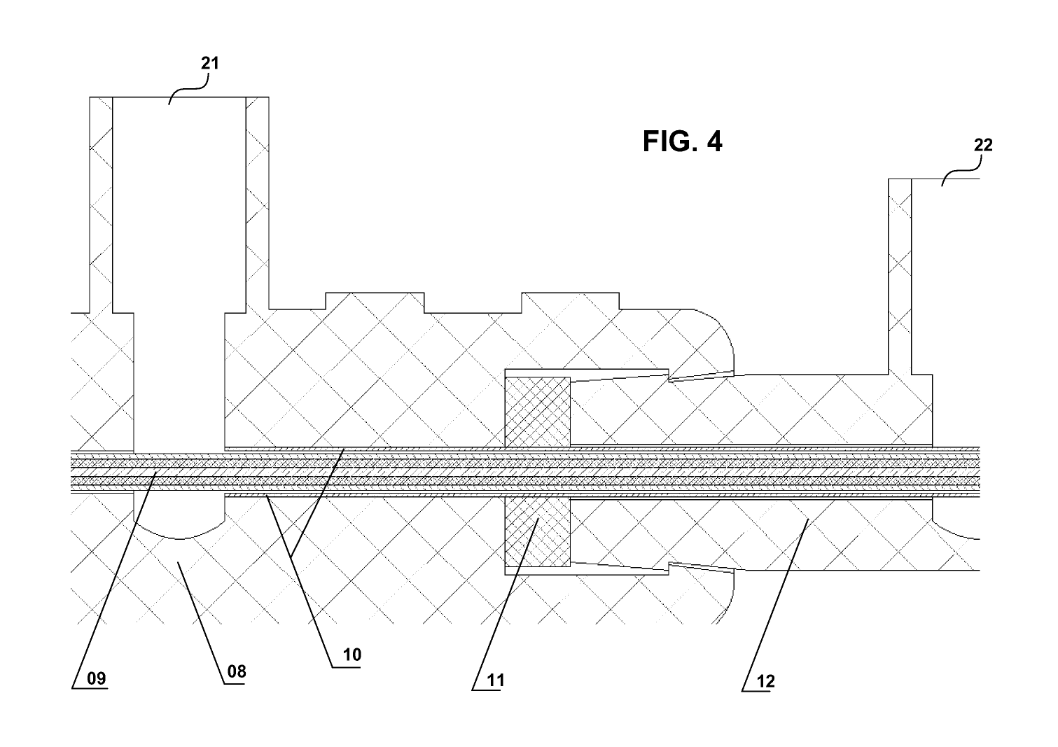

FIG. 4 is a detail view of section 2B in FIG. 2.

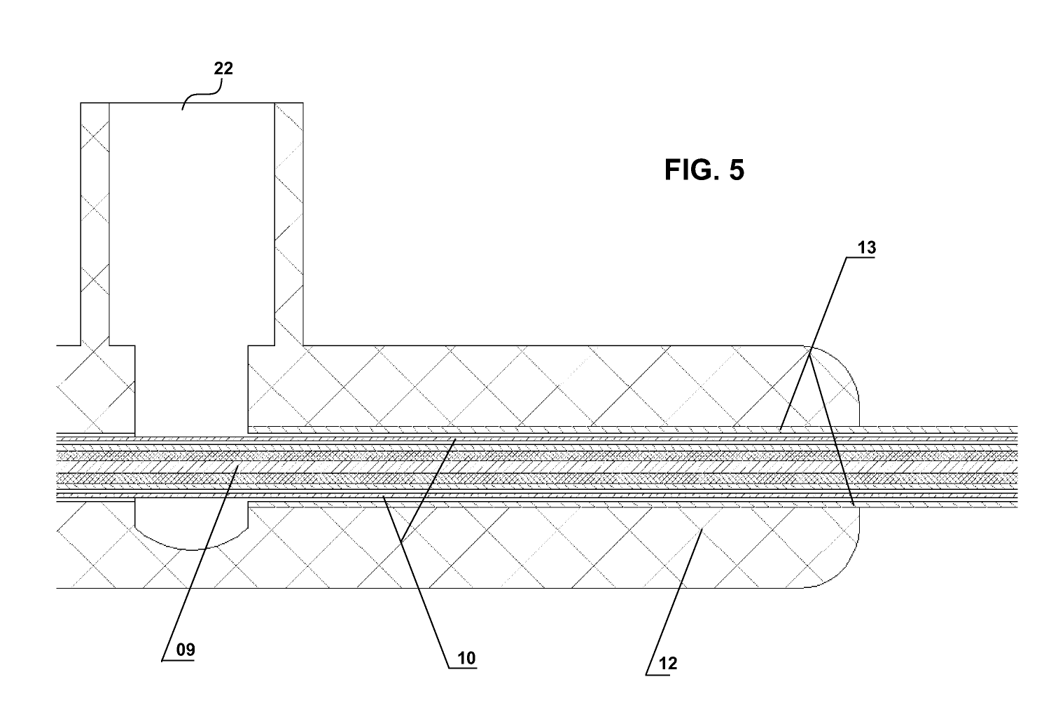

FIG. 5 is a detail view of section 2C in FIG. 2.

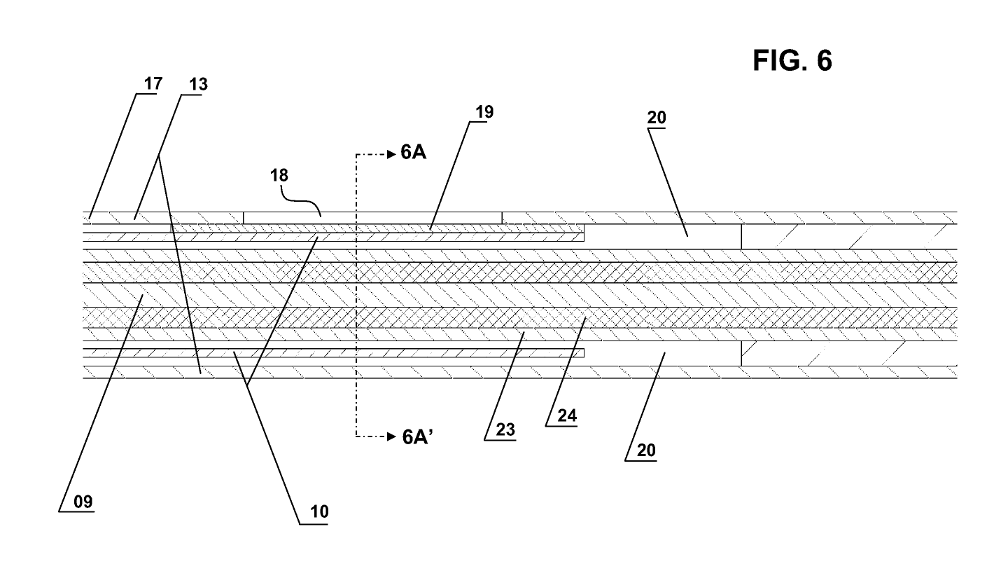

FIG. 6 is a detail view of section 2D in FIG. 2.

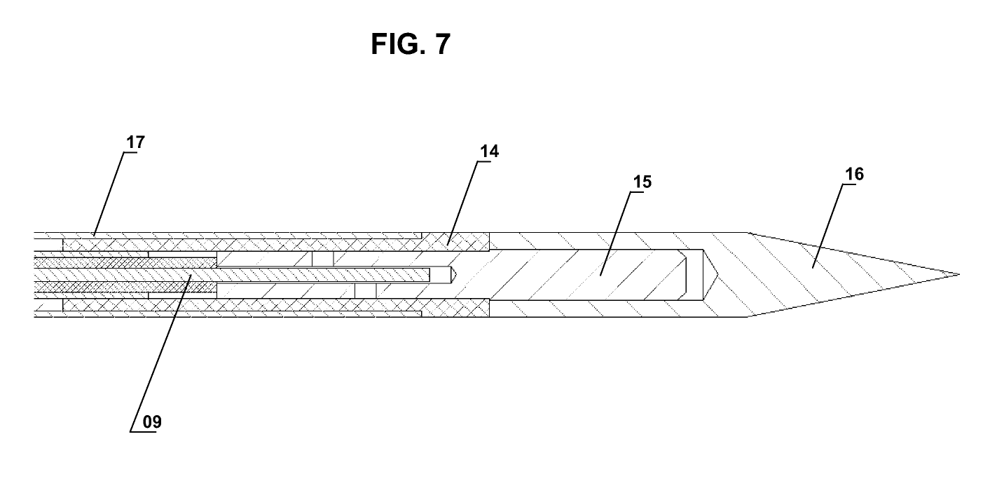

FIG. 7 is a detail view of section 2E in FIG. 2.

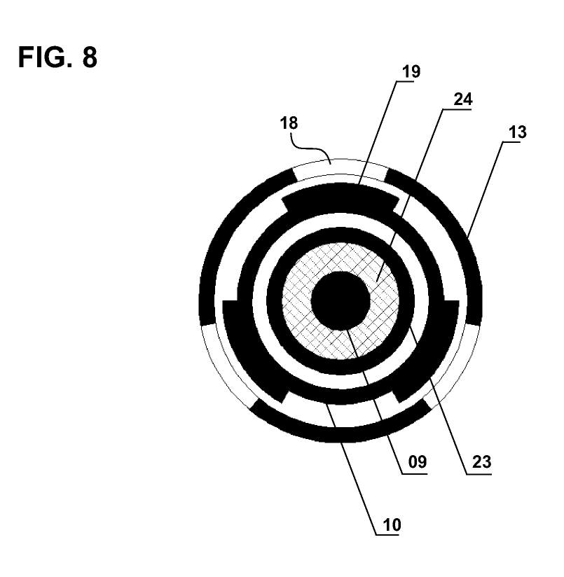

FIG. 8 is a cross sectional view along the line 6A-6A′ in FIG. 6.

### DESCRIPTION OF THE PREFERRED EMBODIMENT

Referring now to FIGS. 1-8, one preferred embodiment of the present invention comprises: the water entry pore (22) and the external pipe (13) on the mold (12) of the external pipe, the medical insulation coating (17) on the surface of the external pipe and the water ejection pores of the external pipe (18), wherein the external pipe (13) is connected to the blade of the microwave scalpel (16) by the weldments (14) and the insulation tube (15); the mold of the external pipe (12) is connected to the mold frame of the internal pipe (08), and there are seal cushion I (11) on the joint surface to prevent water leaking; the water outlet pore (21) and the internal pipe (10) on the mold of the internal pipe (08), wherein the internal pipe (10) is inserted into the external pipe (13) through the seal cushion I (11), and a water storage cavity (20) is formed on the head of the external pipe (13); a stopper (19) on the out surface of the internal pipe (10), so that the stopper (19) can block the water ejection pore (18) as the internal pipe (10) rotates against the external pipe (18) if needs; the mold of the internal pipe (08) is connected to the mold of the electrical outlet (06), and there are seal cushion II (07) on the joint surface to prevent water leaking; the electrical outlet core (01), core seat (02), core seal (03), core cushion (04), and electrical outlet shell (05) on the mold frame of the electrical outlet (06) are together with the microwave conducting wire (09) on the mold frame of the electrical outlet (06) to form the microwave electrode interface; the microwave conducting wire (09) is inside the internal pipe (10) and runs through the whole internal pipe (10), and the microwave conducting wire (09) reaches inside of the isolation tube (15).

During an operation with the above invention, the external pipe (13) rotates against the internal pipe (10) to shift the stopper (19) away from the water ejection pore (18) and allow normal saline inside of the pipes to be ejected into the tissue where the microwave scalpel is working. This produces wetness, infiltration, and heat exchange, and prevents carbonization of the tissue, stops the adhesion of the tissue and extends the ablation area. During the treatment, chemotherapy drugs can also be used with normal saline. While the normal saline is being ejected, the cycle cavity formed between the internal pipe (10) and the external pipe (13) can control the temperature of the tube wall of the microwave scalpel, and prevents the tube wall from scalding surrounding tissues.

It is conceivable that a stopper functions in other ways. For example, instead of rotating against each other, the external pipe and internal pipe can shift along the long axis of the pipes to adjust the position of the stopper against the water ejection pore on the external pipe.

In another preferred embodiment, the external pipe and internal pipe are merged to form a single pipe, and there is no water circulation within the pipe.

While preferred embodiments of the invention have been discussed and illustrated, it should be understood by those of ordinary skill in the art and others that changes can be made to the illustrated embodiments without departing from the spirit and scope of the present invention. Since such changes can be made to the illustrated embodiments while remaining within the scope of the invention, the invention should be defined solely with reference to the appended claims.

What is claimed is:

1. A microwave ablation water jet scalpel for medical operation, comprising:
a microwave scalpel component that comprises
a microwave generating means,
a handle for hand-holding, and
a blade that is attached to one end of said handle; and
a liquid-manipulation component that comprises
a liquid-entry means, and
a liquid-ejection means,
whereby liquid is ejected by said liquid-ejection means to the operating area where said blade is in order to ease the effect caused by heat generated during the operation of said microwave scalpel.

2. A microwave ablation water jet scalpel according to claim 1, wherein said liquid-manipulation component further comprises at least one switch means to control the rate of ejecting liquid to the operating area.

3. A microwave ablation water jet scalpel according to claim 2, wherein the surface of said handle further contains calibrations and medical insulation coating.

4. A microwave ablation water jet scalpel according to claim 2, wherein said handle is a pipe, said liquid-entry means comprises at least one pore on said handle to introduce liquid into said pipe, and said liquid-ejection means comprises at least one pore on said handle to eject liquid from said pipe.

5. A microwave ablation water jet scalpel according to claim 4, wherein the surface of said handle further contains calibrations and medical insulation coating.

6. A microwave ablation water jet scalpel according to claim 1, wherein said handle comprises at least one internal pipe and one external pipe that are substantially concentric, said liquid-ejection means comprise at least one pore on said external pipe to eject liquid from said handle.

7. A microwave ablation water jet scalpel according to claim 6, wherein said liquid-manipulation component further comprises at least one switch means to control the rate of ejecting liquid to the operating area.

8. A microwave ablation water jet scalpel according to claim 7, wherein the surface of said handle further contains calibrations and medical insulation coating.

9. A microwave ablation water jet scalpel according to claim 6, wherein said internal pipe and said external pipe are joined at the end proximate to said blade, said liquid-entry means comprises at least one pore on said handle to introduce liquid into said handle, a liquid-outlet comprises at least one pore on said handle to discharge liquid from said handle, wherein said liquid-entry and said liquid-outlet are arranged so that liquid circulates between the space between said internal pipe and said external pipe and the space inside of said internal pipe.

10. A microwave ablation water jet scalpel according to claim 9, wherein said liquid-manipulation component further comprises at least one switch means to control the rate of ejecting liquid to the operating area.

11. A microwave ablation water jet scalpel according to claim 10, wherein said switch means comprises at least one stopper attached to the outside surface of said internal pipe, and wherein said external pipe is rotatable against said internal pipe, whereby said liquid-ejection means is blocked by said stopper when said external pipe rotates into a position and is unblocked when said external pipe rotates off the position.

12. A microwave ablation water jet scalpel according to claim 11, wherein the surface of said handle further contains calibrations and medical insulation coating.

13. A microwave ablation water jet scalpel according to claim 9, wherein an addition storage cavity connects to the joint between said internal pipe and said external pipe.

14. A microwave ablation water jet scalpel according to claim 13, wherein said liquid-manipulation component further comprises at least one switch means to control the rate of ejecting liquid to the operating area.

15. A microwave ablation water jet scalpel according to claim 14, wherein said switch means comprises at least one stopper attached to the outside surface of said internal pipe, and wherein said external pipe is rotatable against said internal pipe, whereby said liquid-ejection means is blocked by said stopper when said external pipe rotates into a position and is unblocked when said external pipe rotates off the position.

16. A microwave ablation water jet scalpel according to claim 15, wherein the surface of said handle further contains calibrations and medical insulation coating.

17. A microwave ablation water jet scalpel for medical operation, comprising:
A. a microwave generating module that comprises a microwave electrode interface, a conducting wire extending from said interface, and a first assembly frame;
B. an internal module that comprises an internal pipe which contains at least one liquid-outlet, a second assembly frame through which said internal pipe locks into said first assembly frame of said microwave generating module, and a third assembly frame, wherein said conducting wire passes through the inside of said internal pipe;
C. an external module that comprises an external pipe which contains at least one liquid-ejection pore and one liquid-entry pore, a fourth assembly frame through which said external pipe locks into said third assembly frame of said internal pipe, and a fifth assembly frame, wherein a cavity formed between the outside surface of said internal pipe and the inside surface of said external pipe;
D. a scalpel module that comprises a holder, a sixth assembly frame through which said holder locks into said fifth assembly frame of said external pipe, a blade securely attached to said holder, and an insulating tube receiving said conducting wire out from said internal pipe and separating said conducting wire from said blade; and
E. a stopper attached to the outside surface of said internal pipe, wherein said external pipe is rotatable against said internal pipe, whereby said liquid-ejection pore is blocked by said stopper when said external pipe rotates into a position to stop liquid ejecting from inside of the cavity, and whereby said liquid-ejection pore is unblocked when said external pipe rotates off the position to allow liquid ejecting from inside of the cavity.

18. A microwave ablation water jet scalpel according to claim 17, wherein the surface of said handle further contains calibrations and medical insulation coating.
